(19) United States
(12) Patent Application Publication
(10) Pub. No.: US 2005/0028237 A1
(43) Pub. Date: Feb. 3, 2005

(54) Actuator systems

(76) Inventors: Richard Greenhill, London (GB); Hugo Elias, London (GB); Richard Walker, London (GB); Mattew Godden, Luton (GB)

Correspondence Address: RICHARD GREENHILL, 357 LIVERPOOL ROAD, LONDON, N11NL, GB

(21) Appl. No.: 10/926,463

(22) Filed: Aug. 26, 2004

Related U.S. Application Data
(63) Continuation of application No. PCT/GB03/00911, filed on Mar. 4, 2004.

(30) Foreign Application Priority Data
Mar. 7, 2002 (GB) GB 0205339.5

(51) Int. Cl. B25J 18/00; A61F 2/74

(52) U.S. Cl. 901/22; 623/26

(57) ABSTRACT

In an air muscle powered actuator system, notably an anthropomorphic actuator system, a joint 13, incorporates a member 55 with a cylindrical surface; an air muscle 57 which is wrapped around the cylindrical surface such as to produce a flattened portion 61a intermediate two end portions 61b, 61c, to either of which air may be admitted and subsequently exhausted; means 53 clamping the flattened portion to the cylinder such as to prevent migration of air between the end portions; and, extending from the cylindrical member 55, a composite lever structure 67a, 67b. Angular displacement in one sense or the other of the cylinder 55 about an axis parallel to its direction of length, accordingly as air is admitted to one or the other end portion produces corresponding angular displacement in the lever structure 67a, 67b.

Continuation of prior PCT Application No. PCT/GB03/00911 dated 4th Mar. 2004

### BACKGROUND OF THE INVENTION

This invention relates to actuator systems powered by artificial muscles.

The artificial muscle utilized in the arrangements in accordance with the invention is of the kind commonly referred to variously as air muscle, fluidic muscle, rubbertuator, or McKibben muscle.

The artificial muscle which, hereinafter, is referred to as an “air muscle”, comprises: an expansible tubular chamber, generally of an elastomeric material, most commonly rubber, having an air inlet port and an air exhaust port, a common port being, generally, employed for both of these functions; a braided sheath which embraces said tubular chamber throughout its length; and first and second closure arrangements, at the ends, respectively, of the tubular chamber.

The Specification of UK Patent GB No 2255961, dated 13 Mar. 1992, contains a disclosure of a mechanical actuator having an air muscle as above stated, the air muscle serving as actuator traction element.

The air inlet and exhaust porting means of the air muscle may be constituted as a single combined port commonly integral with one or the other of the closure arrangements, but it may be separate from such c).osure arrangement, being, advantageously, a tapping at the mid-length position of the tubular chamber.

Introduction of air, or other suitable fluid, under pressure, to the chamber causes it to expand rapidly, this, in turn, producing radial expansion, also, of the braided sheath.

It is characteristic of the braided sheath, that radial expansion of its expansible tubular chamber is accompanied by a contraction in its length. If the ends of the sheath are respectively coupled, the one to a, possibly fixed, datum, a force-reaction part of the actuation system, the other to a system part movable with respect to said reaction part, contraction of the braided sheath gives rise to a tensile force which acts on the movable system part moving it against reaction at the datum force reaction part in accordance with 10 the extent of contraction in the sheath.

Air muscles need to be pulled out when ‘empty’ (relaxed) in order to be able to deliver their full stroke when inflated. In some cases this extension of the muscle is achieved by a second air muscle coupled to the first, usually acting antagonistically, sometimes by a conventional mechanical spring arrangement or other elastic means which carries out the return movement of a part to be moved. In either circumstance a return movement is effected of the part moved by the air muscle under previous inflation of its tubular chamber.

According to the invention, an actuator system is as set out in the claims of the claims schedule hereof, and said claims and their inter-dependencies are to be regarded as being notionally set out here, mutatis mutandis, also.

### BRIEF DESCRIPTION OF THE DRAWINGS

An embodiment of an actuator system in accordance with the invention is hereinafter described with reference to the accompanying drawings in which:

FIG. 1 is a schematic diagram of an artificial arm/hand skeletal system;

FIG. 2 is a pictorial diagram of the artificial arm/hand skeletal system of FIG. 1;

FIG. 3 is a frontal sectional view of the shoulder joint/upper arm portion of FIG. 1;

FIG. 4 is a pictorial view of the shoulder joint of FIG. 3;

FIG. 5 is a pictorial view of the shoulder joint/upper arm of FIGS. 3 and 4; and,

FIG. 6 is a pictorial view showing the shoulder joint/upper arm of FIG. 5 but with the double muscle thereof removed and other muscles associated with the shoulder joint/upper arm in place.

### DESCRIPTION OF A PREFERRED EMBODIMENT OF THE INVENTION

Whilst the present invention is concerned with an air muscle driven actuation system in which the air muscle is constituted as a double air muscle (as hereinafter described) the invention will be described in the context of a characteristic portion of a humanoid robotic system.

Before entering into a description of such characteristic a robotic system portion embodying the invention, terminology hereinafter employed in relation to several parts od the system will be explained and defined.

(i) Degree of Angular Movement (or Displacement)

When two objects are pivotally connected, the resulting assembly is said to have a degree of angular movement at the pivot. An assembly with N degrees of angular movement is one where there are N pivots within the assembly;

(ii) Universal Joint

This is an assembly connecting two components where the connection has two orthogonal axes of angular displacement.

(iii) Proximal and Distal

The proximal end of a component is the near end, the distal the far end. These terms are used in anatomy where the distal end of a component is the further from the torso, the proximal end the nearer.

(iv) Tendon

A tendon is a flexible tenuous element capable of supporting tensile but not compressive forces. It is used to transmit tensile force between an actuator and a component that the actuator has to move.

To avoid the introduction of fabricated terminology when referring to humanoid robotic parts, the description will employ for such parts the term employed in relation to the human body for the part performing the same function. So, for example, the term humerus will be employed in referring to the robotic part serving, in the robotic system, the function of the human humerus bone.

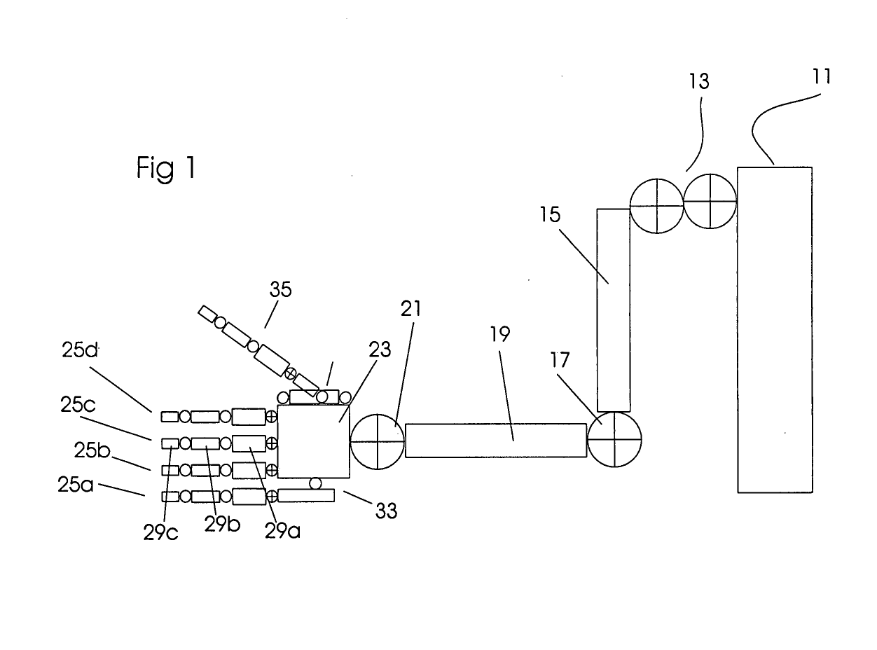

The hand/arm sub-system of a humanoid robotic system comprises (FIG. 1):
(a) a base 11, the equivalent of a local reference frame, being in a humanoid robotic system a robotic part adjacent to the hand/arm sub-system;
(b) a shoulder joint 13;
(c) an upper arm 15, the equivalent of the humerus;
(d) an elbow joint 17;
(e) a forearm 19, incorporating a radius member;
(f) a wrist joint 21, the equivalent of the carpus;
(g) a palm 23
(h) four fingers 25a to 25d, respectively, each having three phalanges, as 29a, 29b, 29c, respectively with joints, as 31a, 31b, respectively, therebetween;
(i) a joint 33 for the raising of the little finger 25a; and,
(j) a thumb 35 having a base joint 37 by which it is attached to the palm 23.

In FIG. 1, shoulder joint 13 is depicted, by the presence of two crossed circles, as having two independent axes of angular movement about independent (orthogonal) axes, the shoulder joint 13 constituting a universal joint, that is to say. The elbow and wrist joints, on the other hand, have each, by analogy with the human arm, a single axis of angular movement.

Whilst FIG. 1 is a synoptic diagram of the overall hand/arm sub-system, the ensuing description focuses on the construction and operation of the shoulder joint/upper arm portion of the skeletal actuator system, where the feature characterizing the present invention is to be found. Apart from the foregoing references, in the text and in the accompanying drawings, to other parts of the hand/arm sub-system the description will address the construction of the shoulder joint and the humerus, only.

The motive power for all parts of the system is provided by air muscles, each having porting means by which pressurized fluid, most conveniently air, may be admitted to and exhausted from the muscle in a controlled manner.

One end of each muscle is attached, most commonly, to a local frame portion, the other (often the distal end), commonly, to an element to be moved, either directly or indirectly, by means of a tendon. Tendons may be routed, using pulley wheels and/or guides, through other parts of the system to the part to be moved. Movement produced by an air muscle may be rectilinear or it may be angular movement about an appropriate axis. In the ensuing description, the movement produced by air muscle actuation is angular displacement, about an axis, at a joint.

Whilst air muscles of the hand/arm sub-system are single muscles, where, as with the shoulder joint, the muscle is to effect rotation of the joint about an axis a double muscle as hereinafter described may, with advantage, be employed.

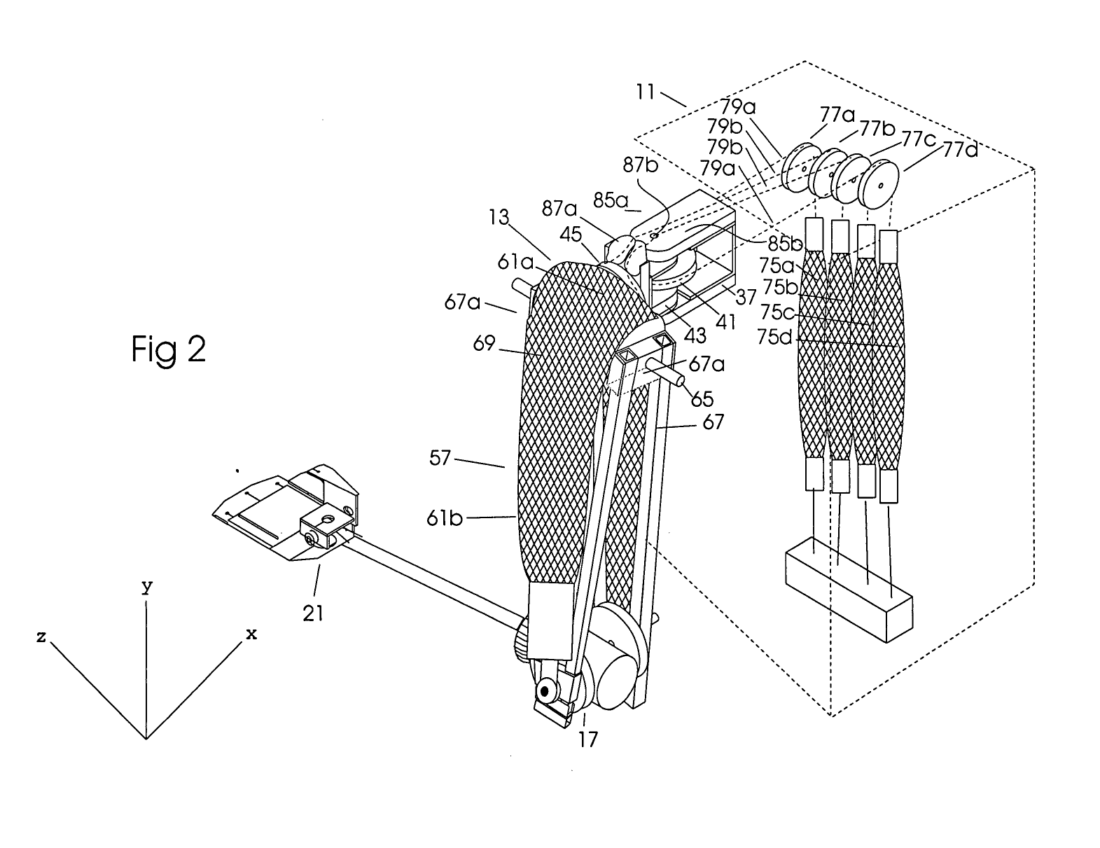

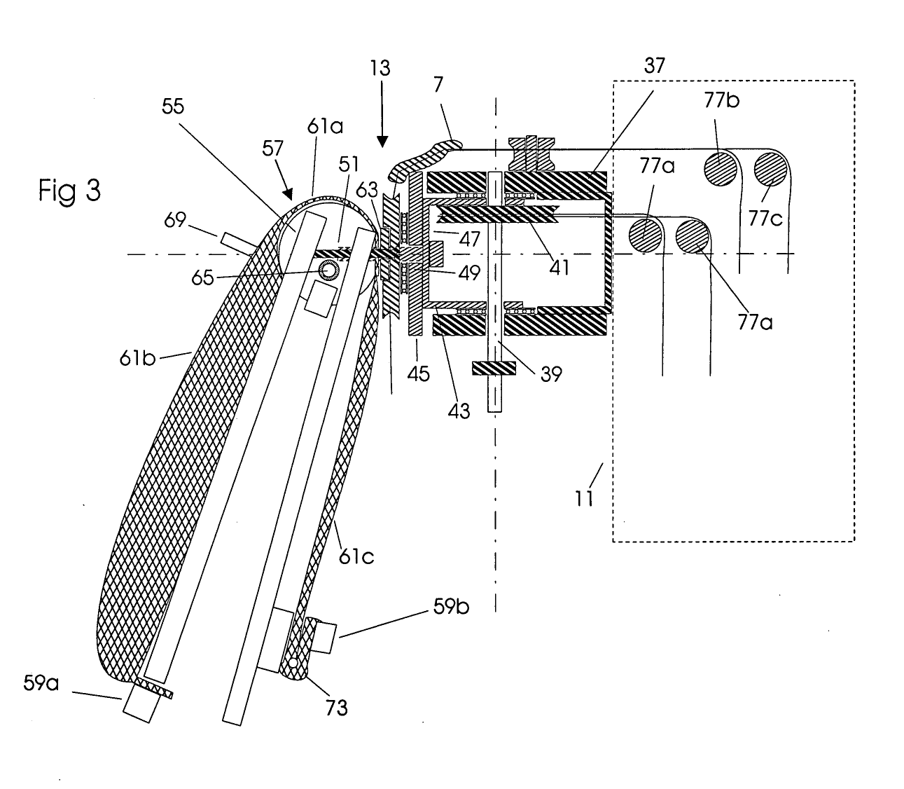

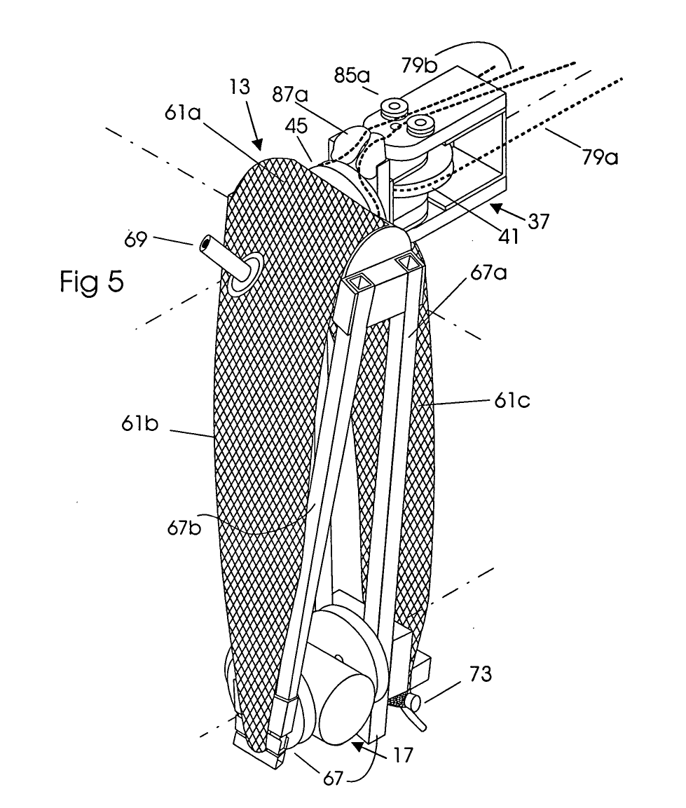

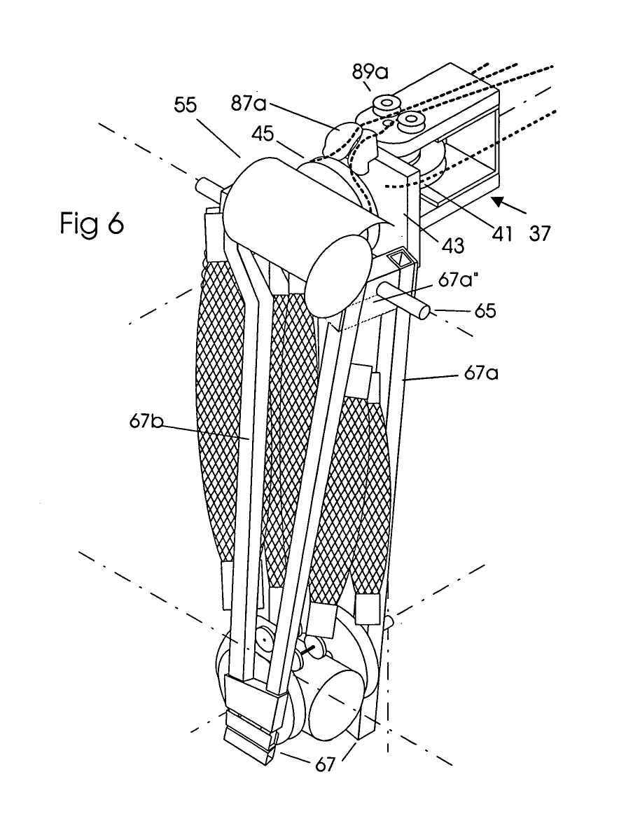

Referring to FIGS. 2 to 6, the shoulder joint 13 has three parts. There is a channel-shaped assembly 37, fixed with respect to the torso, the base 11 that is. The assembly 37 holds a vertical (Y-axis) axle 39. A first pulley wheel 41 is angularly displaceable about the axle 39. A smaller channel-shaped assembly 43 is fixedly attached to the pulley wheel 41. A plate 45, which is secured to the web portion 47 of the channel-shaped assembly 43, has an aperture which receives the spigot end 49 of an horizontal axle 51.

A second pulley wheel 53 which is rotatable about said axle 51 is fixedly attached to a cylinder 55 with the axle 51 extending through an opening therein to intercept the cylinder longitudinal axis intermediate the cylinder ends. With the axle 51 residing parallel to the system X-axis, the cylinder axis resides parallel to the system Z-axis.

An air muscle 57 closed at its ends by first and second closure means 59a, 59b, is wrapped around a substantial peripheral surface portion of the cylinder 55, such as to provide a flattened portion 61a intermediate first and second end portions 61b, 61c, respectively, of the muscle. The flattened intermediate portion 61a is trapped between the cylindrical surface of the cylinder 55 and a clamping bar 63 secured to the pulley wheel 53 and tightly connected thereto by screw connectors (not shown), with the axle 51 extending through an aperture (not shown) through the flattened intermediate portion 61a. The clamping so effected serves to isolate the end portions 61b, 61c, from one another, air being unable during operation to migrate between the end portions 61b, 61c, by way of the intermediate portion 61a. A muscle, constrained as stated above, is, for convenience, ref-erred to a “double muscle”, the two end portions 61b, 61c, each constituting an individual actuation element.

An axle 65 extends lengthwise of the cylinder 55, being offset parallel to and a little below the longitudinal axis of the cylinder. Secured to the cylinder 55 one at each cylinder end 55a, 55b, respectively, there is a lever arrangement or frame structure, 67a, 67b, as the case may be. As may be seen, each of the frame structures 67a, 67b, is in the form of a truncated tetrahedron, top members as 67a′, of the frame structures being respectively attached to the axle 65 at its extremities. The frame structures 67a, 67b, together constitute an upper arm skeletal part, the humerus 67 for brevity.

The end portions 61b, 61c, of the double muscle 57 are respectively attached at their extremities 61b′, 61c′, to the frame structures 67a, 67b at positions remote from the axle 65. Angular displacement of the cylinder 55, as hereinafter described, produces bodily angular movement of the humerus 67 about the off-axis axle 65. The muscle portion 61b of the double air muscle 57 has as its porting means, a tubular member 69 which communicates with the muscle portion interior at a location remote from the header 59a.

The muscle portion 61c on the other hand has as its porting means, a tubular member 73 in communication with the muscle end portion 61c at a position adjacent to the header 59b.

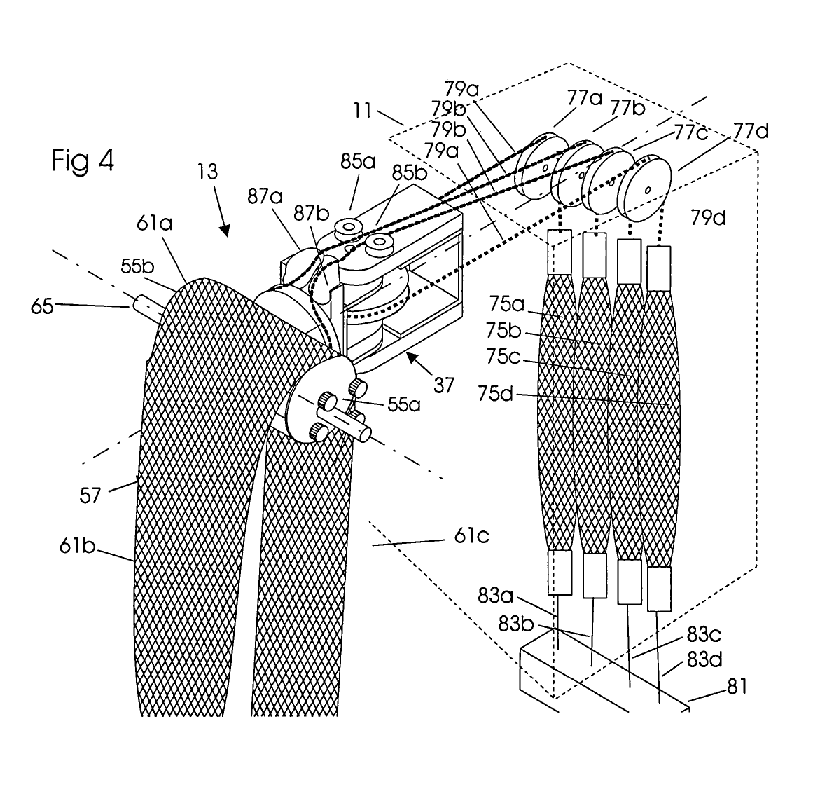

Associated with the base, or torso, 11, for angular movement of the shoulder joint, there are (FIG. 4) four single air muscles, 75a to 75d, respectively; four wheels, 77a to 77d, respectively associated with the muscles 75a to 75d; and two tendons, 79a, 79b, respectively extending between the muscles 75a to 75d, around their respective wheels 77a to 77d, to the joint 13.

The single muscles 75a to 75d, which are connected, at their distal ends, to local reference frame 11, by tendons 81a to 81d, respectively, are associated with one another in pairs, the tendon 79a extending between proximal ends of the paired single muscles 75a, 75d, around and in frictional driving contact with the pulley wheel 41, whilst the tendon 79b extends between the paired single muscles 75b, 75c, by way of guides 85a; 85b, and guides 87a, 87b, around and in frictional driving contact with the pulley wheel 45. Each of the muscles 75a to 75d is, of course, furnished with individual porting means (not shown) for the admission and exhaustion of air from a controlled air pressure source (not shown) for actuating the several muscles. The shoulder joint 13 has at least three degrees of angular movement. These are:
(i) extension/flexion (sideways)
(ii) shoulder rotation (about axis of humerus)
(iii) abduction/adduction (backwards and forwards) Shoulder rotation (about axis of the humerus 67) is effected by different combinations of actuation of the muscles, both single and double, depending upon the degree of extension and flexion of the arm. The muscles 75a, 75d, (hereinafter ‘Vert. Axis muscles’), execute rotation of the humerus about the vertical axis, the other pair 75b, 75c, (‘Horiz. Axis muscles’) effect rotation about a horizontal axis. When the arm is in the orientation shown in FIG. 2 (i.e. humerus 67 hanging vertically), it can be rotated about the axis of the humerus by the action of the Vert.Axis muscles 75a, 75d. When the arm is extended horizontally (i.e. parallel to the X-or Z-axis depending upon the abduction/adduction condition of the arm), the humerus can be rotated by the Horiz.Axis muscles 75b, 75c. Whenever the humerus is not truly horizontal or truly vertical cross-coupling occurs between axes, and both sets of muscles need to be activated in combination, in some measure, in order to effect the desired axial rotation. Different measures of traction of the Vert.Axis and Horiz.Axis muscles, in combination, are, therefore, used to obtain a desired rotation of the humerus 67 about its axis.

In operation, the double air muscle 57, being fixedly attached to the cylinder 55, acts on the cylinder such as to cause it to move in one rotational sense or the other depending upon which of the two muscle end portions 61b, 61c, is inflated, inflation of the portion 61b serving to produce a counter-clockwise angular displacement of the cylinder 55 accompanied by a corresponding extension of the humerus 67a, 67b, whereas inflation of the air muscle portion 61c gives rise to clock-wise rotation of the cylinder 55 accompanied by a corresponding flexion of the humerus.

The off-axis position of the axle 65 improves the leverage available upon extension of the humerus 67. humerus 67a, 67b, such as to cause angular movement of the cylinder 55 in one sense or the other, the sense of angular displacement of the humerus depending upon which of the two end portions 61b, 61c, of the double muscle is inflated, inflation of the portion 61b serving to produce a counter-clockwise rotation in the cylinder 55 accompanied by extension of the humerus 67a, 67b, whereas inflation of the air muscle portion 61c gives rise to clock-wise rotation of the cylinder 55 accompanied by flexion of the humerus 67.

The off-axis position of the axle 65 improves the leverage available upon extension of the humerus 67. The benefits arising from the use of the double air muscle as compared with two single air muscles that might have been employed, are firstly, that most of the construction cost of an air muscle of which, in a humanoid robots and many other applications are numerous is in the headers: the end-closure bung and retaining means, (ring, circlip or other cincture), called for at each end of the muscle, are normally the most expensive items of the assembly. In cost critical applications, cost benefit achieved in the reduction in number, wherever practicable, in a pair of muscles from four to, employing the double air muscle, two may be very substantial.

More important, perhaps, is the matter of space saving. Space occupied by muscles is, as might well be imagined, often at a premium. Any contribution to space available in a muscle rich environment is to be welcomed. Although extremely efficient in terms of power-to-weight ratio, the performance of air muscles in terms of power-to-volume is less impressive. It follows that, in air muscle powered automata, any space saving is valuable. A. notable example arises in connection with the anthropomorphic robot. In this, the air muscles would have to fit into the same or closely similar space as those of a human, a most demanding requirement.

1. An actuator system characterized by:
(a) a local reference frame (11);
(b) joint means (13) supported in bearings with respect to said local reference frame (11) such as to be angularly displaceable about an axis defined in said joint means, and having a convexly curved, two-dimensional surface (51) which extends, widthwise, parallel to said joint axis;
(c) lever means rigidly secured to said joint means such as to be constrained, upon angular displacement of said joint means about said axis, to swing bodily in an arc about said axis in sympathy with the angular displacement about said joint axis;
(d) an air muscle (57) connected at its ends to said lever means and being wrapped about said convexly-curved joint surface with an intermediate portion (61a) thereof, being a portion thereof which is contiguous at its extremities with end-portions (61b, 61c) of said air muscle, and being deformed to a flattened state, as a result of its contact with said convexly-curved joint surface, across the full width of said muscle and over the full length of contact between said intermediate muscle portion and said joint surface;
(e) first and second air admission and exhaust porting means, being porting means communicating, respectively, with the air muscle interior at locations thereof within said air muscle end portions (61b, 61c); and,
(f) clamping means (63) serving to clamp said flattened intermediate muscle portion (61a) to said joint along a fully widthwise-extensive section of said intermediate muscle portion such as to isolate said muscle end-portions (61b, 61c) against migration of air therebetween.

2. An actuator system as claimed in claim 1 characterized in that the spacing between said convexly curved joint surface (51) and said joint axis (65) is such that the rate of change of length of radial vectors between said axis and said surface increases and decreases with angle in a smooth continuous manner about said axis, being at a maximum at an intermediate angular position.

3. An actuator system as claimed in claim 1 or 2 characterized in that said convexly-curved surface (51) is a cylindrical surface.

4. An actuator system as claimed in claims 2 and 3 characterized in that said joint axis (65) is not coincident with the longitudinal axis of said cylindrical surface.

5. An artificial limb system which comprises an actuator as claimed in claim 1, characterized in that said lever part (57) comprises a skeletal human limb part.

6. An artificial limb system as claimed in claim 5 characterized in that said skeletal limb part corresponds to the humerus bone.
